(19) United States
(12) Patent Application Publication
(10) Pub. No.: US 2014/0050810 A1
(43) Pub. Date: Feb. 20, 2014

(54) ANTI-OBESITY COMPOSITION COMPRISING LYCIUM CHINENSIS LEAF EXTRACT AND BETAINE AS ACTIVE INGREDIENT

(71) Applicant: BIONUTRIGEN CO., LTD., Taejon (KR)

(72) Inventors: Song Hae BOK, Daejeon (KR); Eun Eai KIM, Daejeon (KR); Jae Yeol YOO, Daejeon (KR); Myung-Sook CHOI, Daegu (KR)

(73) Assignee: BIONUTRIGEN CO., LTD., Taejon (KR)

(21) Appl. No.: 13/901,861

(22) Filed: May 24, 2013

(30) Foreign Application Priority Data
Aug. 14, 2012 (KR) 10-2012-0089048

(51) Int. Cl. A61K 36/815; A61K 31/205

(52) U.S. Cl. CPC A61K 36/815; A61K 31/205
USPC 424/774; 562/575

(57) ABSTRACT

Disclosed are betaine and a Lycium chinensis leaf extract and a mixture thereof which are preventive and therapeutic of obesity, and uses thereof. They are found to have prophylactic and therapeutic effects on obesity and obesity-induced metabolic syndrome as evaluated for reductive activity against weight, body fat, total cholesterol, triglyceride, and LDL in humans and in mice. Thus, they can be used as active ingredients in pharmaceutical or health functional food compositions for the prevention and treatment of obesity or obesity-induced metabolic syndrome.

### BACKGROUND OF THE INVENTION

1. Field of the Invention

The present invention relates to an anti-obesity composition comprising a Lycium chinensis leaf extract and a betaine compound.

2. Description of the Related Art

Metabolic syndrome is a combination of medical disorders that is characterized by three of obesity, central obesity, raised blood pressure, hypertriglyceridemia, low-HDL cholesterolemia, and raised blood glucose level occurring together. Metabolic syndrome is particularly associated with obesity, and some studies have shown the prevalence in Korea to be an estimated 30% of the population, with 50% of the Korean population older than the age of 65 being affected by metabolic syndrome.

All of the risks of metabolic syndrome stem from obesity, and so the syndrome is difficult to treat because it is deeply associated with dietary habit. It is difficult to expect an improvement in a patient suffering from metabolic syndrome until the patient changes his or her life habits to adopt a proper physical exercise and diet. Meanwhile, there are few medications found to have excellent anti-obesity effects without inducing side effects. In the field of nutrition, few foodstuffs with distinct anti-obesity effects have been found, either. Anti-obesity food compositions, or health functional foods preventive and therapeutic of obesity-induced metabolic syndrome, are typically designed to comprise foods that produce feelings of satiety, with low calories, or safety-guaranteed herbal materials supplemented with dietary fibers, phenolic compounds, flavonoids,

Lycium chinensis is a species of boxthorn in the family Solanaceae, and grows naturally as well as being cultivated in Korea, China, Taiwan, Japan, and Europe. Its seeds, leaves and roots are used in herbal medicines.

Betaine is found in abundance in plants and animals and is biosynthesized through irreversible oxidation of choline. Helping the body to metabolize homocysteine, betaine is approved by the US Food and Drug Administration (FDA) to treat homocysteinemia, a condition where too much homocysteine builds up in the body. Betaine was disclosed to lower homocycteine levels in WO 98/19690 while WO 2000/51596 describes the antithrombotic activity of betaine (U.S. Pat. No. 7,867,525 [symbol] , EP No. 1855549).

Korean Patent Laid-Open Publication No. 10-2004-0095948 discloses an anti-obesity composition comprising a hot water extract of Lycium chinensis leaves. Also, Korean Patent Laid-Open Publication No. 10-2004-0095947 discloses a composition for weight loss, comprising a Lycium chinensis leaf powder and a ginseng powder. There are no reports on the use of a Lycium chinensis powder, or a combination of a hot water extract of Lycium chinensis leaves and a certain compound, such as vitamin C or betaine in inducing weight loss or in improving health. Further, therapeutic and preventive effects of Lycium chinensis extracts or betaine on obesity-induced metabolic syndrome are not disclosed at all.

### SUMMARY OF THE INVENTION

It is therefore an object of the present invention to provide a mixture in which a Lycium chinensis leaf extract and betaine are mixed at as optimal a ratio as to prevent and treat obesity-induced metabolic syndrome.

It is another object of the present invention to provide a pharmaceutical composition for the prevention and treatment of obesity or obesity-induced metabolic syndrome, comprising a Lycium chinensis leaf water extract (LWE) and betaine (hereinafter referred to as “JBB21”) at an optimal ratio as an active ingredient.

It is a further object of the present invention to provide a health function food composition for improving obesity or obesity-induced metabolic syndrome, comprising an (LWE and JBB21 at an optimal ratio as an active ingredient.

In the present invention, an LWE powder and a betaine represented by the following Chemical Formula 1 are evaluated for anti-obesity activity after they are separately administered at various doses. In addition, a formulation ratio between the LWE powder and betaine which is suitable for guaranteeing anti-obesity activity for the mixture of LWE powder and betaine is determined. Also, the prophylactic and therapeutic effect of the mixture on obesity or metabolic syndrome is evaluated in vivo by administering it to mice in which obesity and metabolic syndrome has been induced by a high fat diet. The mixture is prepared into a health functional food for improving obesity or obesity-induced metabolic syndrome or a pharmaceutical composition for preventing and treating obesity or obesity-derived metabolic syndrome.

(CH₃)₃N⁺—CH₃—COO⁻ <Chemical Formula 1>

In accordance with an aspect thereof, the present invention provides a pharmaceutical composition for the prevention and treatment of obesity or metabolic syndrome, comprising a Lycium chinensis leaf extract (LWE) and betaine (JBB21), alone or in combination, as an active ingredient.

In accordance with another aspect thereof, the present invention provides a health functional food composition for improving obesity or metabolic syndrome, comprising a Lycium chinensis leaf extract (LWE) and betaine (JBB21), alone or in combination, as an active ingredient.

In accordance with a further aspect thereof, the present invention provides an anti-obesity food or beverage which can be readily prepared from a commercially available food using the Lycium chinensis leaf extract and betaine, either individually or in combination.

### BRIEF DESCRIPTION OF THE DRAWINGS

The patent or application file contains at least one color drawing. Copies of this patent or patent application publication with color drawing will be provided by the USPTO upon request and payment of the necessary fee.

The above and other objects, features and other advantages of the present invention will be more clearly understood from the following detailed description taken in conjunction with the accompanying drawings, in which:

FIG. 1 shows microscopic images of fatty liver tissues of mice fed with a high-fat diet after administration with a Lycium chinensis leaf extract (LWE), betaine (JBB21), and a mixture thereof (JBB21+LWE).

### DESCRIPTION OF THE PREFERRED EMBODIMENTS

The present invention addresses a composition for the prevention and treatment of obesity or metabolic syndrome, comprising a mixture of a Lycium chinensis leaf powder or an Lycium chinensis leaf hot water extract (LWE) powder plus betaine as an active ingredient.

In one embodiment of the present invention, the betaine may be in the form of a betaine anhydride, or a betaine chloride or any salt of betaine that can release betaine when it is dissolved in water. A composition with betaine, whether natural or synthetic, falls within the scope of the present invention.

Most preferably, a Lycium chinensis leaf extract and betaine are used in mixture because, as will be explained in the following Example section, the ingredients in mixture exhibit a synergistic effect on the prevention and improvement of obesity or metabolic syndrome. An LWE powder and a betaine powder may be mixed at various ratios and preferably at a ratio of 1˜5:1 as shown in Table 1, below.

TABLE 1
Formulations of LWE Powder and Betaine Powder (wt ratios)

| | LWE Powder (wt) | Betaine Powder (wt) |
|---|---|---|
| Control 1 | 1 | 0 |
| Control 2 | 0 | 1 |
| Test 1 | 1 | 1 |
| Test 2 | 2 | 1 |
| Test 3 | 5 | 1 |

In the present invention, a Lycium chinensis powder may be obtained by washing fresh leaves with water, air drying the cleaned leaves, and pulverizing the dried leaves into a size of 60 mesh or less. The Lycium chinensis hot water extract (LWE) powder may be prepared by immersing dried Lycium chinensis leaves in hot water of 90° C.˜105° C. for 4˜8 hrs, concentrating the exudate to the dryness, and pulverizing the dried exudates. In addition, an ethanol extract may be employed. In this case, dried Lycium chinensis leaves are extracted with 60˜95% ethanol, and then the ethanol is evaporated, followed by powdering the residue. Alternatively, a powder may be obtained by grinding fresh Lycium chinensis leaves, extracting the leaf juice in hot water, and powdering the extract, or compressing fresh Lycium chinensis leaves instead of grinding.

The pharmaceutical composition for the prevention and treatment of obesity or metabolic syndrome may be administered orally or parenterally in a general dosage form. It may be formulated together with a diluent or excipient such as a filler, a thickener, a binder, a wetting agent, a disintegrant, a surfactant, etc. For oral administration, the pharmaceutical composition may be formulated into a tablet, a capsule, a pill, a powder, or a granule. As a liquid intended for oral administration, the composition may take the form selected from among a suspension, an internal use liquid an emulsion, and a syrup.

According to an embodiment of the present invention, the mixture of a Lycium chinensis extract and betaine may further comprise another anti-obesity ingredient. For example, tannin, catechin, or hydroxycitric acid (found in a variety of tropical plants including Garcinia cambogia), may be added to the mixture. These compounds are illustrative, but are not intended to limit the present invention.

A better understanding of the present invention may be obtained through the following examples which are set forth to illustrate, but are not to be construed as limiting the present invention.

### Example 1

### Assay for Anti-Obesity Activity of Betaine and Lycium chinensis Leaf Extract Powder

In order to evaluate the anti-obesity effect of Lycium chinensis leaf extract powder and betaine, subjects' weight, body mass index (BMI), body fat, body fat percentage, and muscle mass were measured prior to the administration of betaine and Lycium chinensis leaf extract powder. Betaine and Lycium chinensis leaf extract powder were separately dissolved in water and administered at a single dose of 3 g and 10 g, respectively, twice a day just before breakfast and supper.

In consideration of their component properties, the Lycium chinensis leaf extract powder, which comprised various components, was administered for 22 days while 43 days was also allocated for the administration of betaine, a single compound. Preventive and therapeutic effects of the two ingredients on obesity were evaluated in view of changes in weight, BMI, body fat, body fat percentage, and muscle mass.

The measurements are summarized in Table 2, below.

TABLE 2
Anti-Obesity Effect of Betaine and Lycium
chinensis leaf Extract in Human Body
Betaine 3 g × twice/Day
Lycium chinensis leaf extract powder 10 g × twice/Day

| | Before | | After | | | | | |
|---|---|---|---|---|---|---|---|---|
| | Administration | | Administration | | Change (Kg) | | Reduction (%) | |
| | | L. chinensis | | L. chinensis | | L. chinensis | | L. chinensis |
| | | leaf | | leaf | | leaf | | leaf |
| Test | | extract | | extract | | extract | | extract |
| Item | Betaine | powder | Betaine | powder | Betaine | powder | Betaine | powder |
| Weight | 87.0 | 79.9 | 84.0 | 79.3 | −3.0 | −0.6 | 3.5 | 0.75 |
| (kg) | | | | | | | | |
| BMI | 30.1 | | 29.3 | | −0.8 | −0.1 | 2.7 | |
| Body | 27.6 | 21.0 | 25.9 | 19.6 | −1.7 | −1.4 | 6.2 | 6.67 |
| Fat | | | | | | | | |
| Body | 31.7 | 26.4 | 30.7 | 24.7 | −1.0 | −1.7 | 3.2 | 6.43 |
| Fatness | | | | | | | | |
| Muscle | 56.2 | 55.7 | 55.5 | 56.5 | −0.7 | −0.8 | 1.3 | 1.43 |
| Mass | | | | | | | | |

As can be seen in Table 2, the subjects reduced in body fat by 6.2% when administered with betaine for 43 days and by 6.7% when administered with Lycium chinensis leaf extract powder for 22 days. Data of Table 2 indicate that both Lycium chinensis leaf extract powder and betaine have anti-obesity effects in view of weight, BMT, body fat, and body fat percentage.

### Example 2

### Assay for Anti-Obesity Activity of Composition Comprising Mixture of Betaine and Lycium chinensis Leaf Extract in Human Body

After a mixture comprising 10 wt % of a Garcinia cambogia extract, 10 wt % of a tangerine peel containing bioflavonoids such as hesperidin, naringin, etc., 25 wt % of a Lycium chinensis leaf extract containing tannin, 25 wt % of betaine, 10 wt % of taurine, 10 wt % of choline, 2 wt % of L-carnitine, 1 wt % of vitamin complex (vitamin B12 2 mg, and vitamin A, B1, B2, B6, E, nicotinic acid amide, pantothenic acid, calcium, folic acid, each 20 mg), and 5 wt % of a green tea extract (catechin) was orally administered at a single dose of 6 g twice a day for 25 days, blood samples were taken, and lipid metabolisms were evaluated. A reduction was made in total cholesterol by 20%, in total triglyceride by 41%, in LDL cholesterol by 15%, in GOT by 14%, in GPT by 39%, in rGTP by 38%, in glucose by 11%, in weight by 2 kg (2.4%), and in BMI by 0.3%, indicating a general improvement in obesity, triglyceride level, and fatty liver.

Hence, the composition comprising a mixture of a Lycium chinensis leaf extract and betaine as active ingredients was effective for improving obesity, fatty liver, and lipid metabolism.

### Example 3

### Assay for Anti-Obesity Activity of Composition Comprising Mixture of Betaine and Lycium chinensis Leaf Extract in Animal

A betaine powder, and a Lycium chinensis leaf extract powder were orally administered individually or in mixture to mice the weight and body fat of which were measured to evaluate the preventive and therapeutic effects of them on obesity or metabolic syndrome. In this experiment, the betaine powder and the Lycium chinensis leaf extract powder were used separately, or mixed at the ratio shown in Table 1.

The Lycium chinensis leaf extract powder was prepared as follows. First, 100 kg of Lycium chinensis leaves was cleansed with water, and dried at room temperature for one week in a shaded place. Then, they were immersed in 1000 L of water and extracted at 80˜100° C. for 8 hrs. The extract was dried with hot air and finely pulverized to give 30 kg of a powder.

### Experimental Example 1

### Preparation of Test Animal

Betaine (JBB21), a Lycium chinensis hot water extract powder (LWE), or a mixture thereof (JBB21+LWE) was orally administered five times a week to high-fat diet-induced obesity models and their effects on obesity, hyperlipidemia and fatty liver were analyzed. JBB21 and LWE were purchased as white and brown powders, respectively, from Bionutrigen, and stored at room temperature before use in the experiment.

C57BL/6, specific pathogen-free (SPF) mice (male, six weeks old, Division of Biomedical Research, Korea Research Institute of Bioscience and Biotechnology) were used as the test animals.

The mice were allowed to have access to radiation-sterilized solid foods (15 Kg, Harlan, supplied by Polas International) ad libitum during an acclimation and quarantine period.

For comparison, a negative control and an obese group (high fat diet, HFD) were allowed to freely eat a radiation-sterilized control diet (CD, 10% kcal, Research Diets) and a high fat diet (HFD, 60% Kcal, Research Diets, supplied by Central Lab. Animal), respectively, during the administration of JBB21, LWE, or JBB21+LWE.

JBB21, LWE, and JBB21+LWE were dissolved in 3.D.W at concentrations of 100 mg/mL, 200 mg/mL, and 100+200 mg/mL, respectively, and each of the solutions was orally administered at a dose of 10 mL/kg five times a week for 56 days.

### Experimental Example 2

### Weight Change by Mixture of Betaine and Lycium chinensis leaf Extract Powder

The negative control (CD), the obese group (HFD), and the test groups (JBB21, LWE, and JBB21+LWE) were monitored three times a week for weight change and diet intake before and during administration.

Each of the solutions of JBB21 and LWE was administered at a single dose of 10 mL/kg five times a week for 56 days. The JBB21 (1 g/kg) group was observed to decrease in weight from the 39^{th} day and the 44^{th} day, with statistical significance compared to the obese group, and had experienced 9.4% weight loss by the last day (p<0.01). The LWE (2 g/kg) group decreased in weight by 6.5% by the last day, but no significant changes in weight had been observed during the test period. The JBB21+LWE (1+2 g/kg) group, continued to decrease in weight with significance from day 37, and was measured to have experienced a 10.4% (p<0.01) weight loss by the last day of the experiment.

Accordingly, JBB21 and LWE were more effective for weight loss when administered together than alone, showing a synergistic effect.

TABLE 3
Effect of Betaine and Lycium chinensis leaf
extract Powder on Weight Loss of Animal Model

| | | Dose | Days after treatment | | | | | | |
|---|---|---|---|---|---|---|---|---|---|
| Group (n = 10) | | (g/kg) | 0 | 11 | 21 | 30 | 39 | 51 | 56 |
| Normal | Control | | 25.2 ± 2.0 | 27.5 ± 2.0 | 28.5 ± 2.0 | 29.1 ± 2.4 | 30.6 ± 2.8 | 30.9 ± 2.7 | 31.2 ± 2.8 |
| Fat Diet | | | | | | | | | |
| (10% Kcal) | | | | | | | | | |
| High | V.C | 0 | 31.0 ± 1.5 | 33.8 ± 1.7 | 36.6 ± 2.5 | 39.5 ± 2.5 | 42.5 ± 2.9 | 45.3 ± 3.1 | 46.4 ± 2.8 |
| Fat Diet | (3.D.W) | | | | | | | | |
| (60% Kcal) | JBB21 | 1 | 31.1 ± 1.5 | 31.9 ± 1.6 | 34.5 ± 1.8 | 36.1 ± 2.3 | 38.4 ± 2.7 | 40.9 ± 3.1 | 42.0 ± 3.4 |
| | | IR(%) | | 5.6 | 5.8 | 8.8 | 9.6* | 9.6* | 9.4** |
| | LWE | 2 | 31.1 ± 1.5 | 32.0 ± 2.0 | 34.5 ± 2.7 | 36.3 ± 4.0 | 38.6 ± 3.9 | 41.9 ± 4.2 | 43.4 ± 4.1 |
| | | IR(%) | | 5.4 | 5.9 | 7.4 | 9.2 | 7.6 | 6.5 |
| | JBB21 + | 1 + 2 | 31.1 ± 1.4 | 32.2 ± 1.1 | 34.7 ± 1.7 | 36.6 ± 2.0 | 38.4 ± 2.8 | 40.6 ± 3.8 | 41.5 ± 3.6 |
| | LWE | IR(%) | | 4.8 | 5.3 | 7.4 | 9.6* | 10.4** | 10.4** |

*p < 0.05,
**p < 0.01,
***p < 0.001,
****p < 0.0001 vs. Vehicle Control

During the administration of the test materials, changes in feed intake of the mice were monitored by measuring the amounts of feed that they ate, three times a week. The results are summarized in Table 4, below. As can be seen in Table 4, significant changes in feed intake were detected in none of the negative control (CD), the obese group (HFD), the JBB21 (1 g/kg) group, the LWE (2 g/kg) group, and the JBB21+LWE (1+2 g/kg) group.

TABLE 4
Feed Intake of Mice Administered with Lycium chinensis
leaf extract Powder and/or Betaine Powder

| | | Dose | Days after treatment | | | | | | |
|---|---|---|---|---|---|---|---|---|---|
| Group (n = 10) | | (g/kg) | 11 | 21 | 30 | 39 | 51 | 56 | 39 |
| Normal | Control | | 3.99 ± 0.48 | 3.72 ± 0.29 | 3.49 ± 0.26 | 4.18 ± 0.55 | 3.69 ± 0.33 | 3.71 ± 0.46 | 4.18 ± 0.55 |
| Fat Diet | | | | | | | | | |
| (10% | | | | | | | | | |
| Kcal) | | | | | | | | | |
| High | V.C | 0 | 2.91 ± 0.84 | 3.29 ± 0.64 | 3.59 ± 0.19 | 3.16 ± 0.15 | 3.94 ± 0.62 | 3.37 ± 0.40 | 3.16 ± 0.15 |
| Fat | (3.D.W) | | | | | | | | |
| Diet | JBB21 | 1 | 3.32 ± 0.86 | 2.93 ± 0.98 | 3.59 ± 0.19 | 3.16 ± 0.15 | 3.94 ± 0.62 | 3.37 ± 0.40 | 3.16 ± 0.15 |
| (60% | | IR(%) | −14.2 | 10.8 | 15.7 | 10.4 | 27.1 | −0.5 | 10.4 |
| Kcal) | LWE | 2 | 3.08 ± 0.90 | 2.93 ± 0.98 | 3.03 ± 0.96 | 3.26 ± 0.55 | 3.89 ± 0.47 | 3.31 ± 0.25 | 3.26 ± 0.55 |
| | | IR(%) | −5.9 | 10.8 | 15.7 | −2.9 | 1.4 | −2.0 | −2.9 |
| | JBB21 + | 1 + 2 | 2.70 ± 0.75 | 3.24 ± 0.48 | 3.36 ± 0.34 | 3.03 ± 0.26 | 3.51 ± 0.52 | 3.25 ± 0.25 | 3.03 ± 0.26 |
| | LWE | IR(%) | 7.1 | 1.5 | 6.5 | 4.1 | 11 | 0 36 | 4.1 |

*p < 0.05,
**p < 0.01,
***p < 0.001,
****p < 0.0001 vs. Vehicle Control

Meanwhile, subcutaneous fat was also measured. As can be seen, subcutaneous fat was decreased by 14.2% in the JBB21 (1 g/kg) group and by 14.1% in the LWE (2 g/kg) group, compared to the obese group. When administered with JBB21+LWE (1+2 g/kg), the mice decreased in subcutaneous fat by 32.7% (p<0.001), which was 2.3 times more than the decrement of the JBB21 group or the LWE group, indicating that co-administration of JBB21 and LWE has a synergistic effect, compared to individual administration of JBB21 and LWE.

TABLE 5
Reductive Effect of Betaine and/or Lycium chinensis leaf
extract Powder on Subcutaneous Fat in Animal Model

| | Control | High Fat Diet (60% Kcal) | | | |
|---|---|---|---|---|---|
| | Diet | | | | JBB21 + |
| | (10% | V.C | JBB21 | LWE | LWE |
| Group | Kcal) | (3.D.W) | (1 g/kg) | (2 g/kg) | (1 + 2 g/kg) |
| Subcutaneous | 848.5 ± 291.7 | 3467.1 ± 420.8 | 2975.4 ± 530.4 | 2977.0 ± 585.8 | 2332.1 ± 727.0 |
| Fat(mg) | | | | | |
| IR(%) | | | 14.2 | 14.1 | 327*** |
| Total | 1973.3 ± 783.9 | 7349.3 ± 644.5 | 7030.2 ± 947.9 | 7044.1 ± 1015.7 | 6069.3 ± 1408.4 |
| Fat(mg) | | | | | |
| IR(%) | | 4.3 | 4.2 | 17.4* | |

*p < 0.05,
**p < 0.01,
***p < 0.001,
****p < 0.0001 vs. Vehicle Control

In addition, total fat was measured in the mice administered with JBB21, LWE, or JBB21+LWE. Compared to the obese group, total fat was found to decrease by 4.3% in the JBB21 (1 g/kg) group, and by 4.2% in LWE (2 g/kg) group. Further, the total fat of the JBB21+LWE (1+2 g/kg) group was reduced by 17.4% (p<0.05), which was four times more than the decrement in the group administered with JBB21 or LWE alone.

Hence, co-administration of JBB21+LWE exhibited a synergistic effect on the reduction of total fat as well as body fat, compared to individual administration of JBB21 or LWE.

### Experimental Example 3

### Biochemical Change of Blood by Betaine and Lycium chinensis Leaf Extract Powder

Blood samples taken on 56 days after the administration of a mixture of JBB21 and LWE were placed in heparin-coated tubes and stored on ice, followed by centrifugation at 6,000 rpm for 5 min. The plasma thus obtained was analyzed for levels of blood leptin, a hormone marker for obesity, adiponectin, an anti-obesity hormone, and triglyceride levels, using an automated biochemistry analyzer (AU400, Olympus, Japan). Leptin and adiponectin vary in secretion and function depending on the level of fat accumulated in the body. The preventive and therapeutic effects of the mixture according to the present invention were determined in view of plasma levels of leptin and adiponectin.

In this regard, the blood levels of the hormones were measured using Mouse Leptin ELISA kit (Cat. #: MOB00) and Mouse Adiponectin ELISA kit (Cat. #: MRP300), respectively, both commercially available from R&D Systems.

The blood leptin level of the obese group was 5.8 times higher than that of the control group. Compared to the obese group, the JBB21 (1 g/kg) group and the LWE (2 g/kg) group decreased in blood leptin level by 9.1% and 12.9%, respectively. The blood leptin level of the JBB21+LWE (1+2 g/kg) group was reduced by 28.3% (p<0.05), which is greater than two times the decrement of the individually administered groups, indicating a synergistic effect of JBB21 and LWE (Table 6).

In addition, the synergistic anti-obesity effect of the present invention was demonstrated as the anti-obesity hormone adiponectin was further secreted upon the co-administration of JBB21 and LWE.

TABLE 6
Blood Leptin and Triglyceride Levels of Animal Models Administered
with Betaine and/or Lycium chinensis leaf extract Powder

| | | High Fat Diet (60% Kcal) | | | |
|---|---|---|---|---|---|
| | | | | | JBB21 + |
| | Control | | | | LWE |
| | Diet(10% | V.C | JBB21 | LWE | (1 + 2 |
| Group | Kcal) | (3.D.W) | (1 g/kg) | (2 g/kg) | g/kg) |
| Leptin | 25.3 ± | 147.9 ± | 134.3 ± | 128.8 ± | 106.0 ± |
| (ng/ml) | 19.0 | 24.5 | 41.7 | 34.1 | 46.1 |
| IR(%) | | | 9.2 | 12.1 | 28.3* |
| Triglyceride | 68.8 ± | 96.2 ± | 74.7 ± | 87.1 ± | 54.1 ± |
| (mg/dl) | 18.4 | 48.5 | 26.3 | 54.1 | 15.8 |
| IR(%) | | | 22.3 | 9.5 | 43.8* |

*p < 0.05,
** p < 0.01,
***p < 0.001,
****p < 0.0001 vs. Vehicle Control

As for triglyceride, its blood level was reduced by 22.3% in the JBB21 (1 g/kg) group and by 9.5% in the LWE (2 g/kg) group, compared to the control group, but with no statistical significances. However, the blood triglyceride level of the JBB21+LWE (1+2 g/kg) group was synergistically reduced by 43.8% with a statistical significance (p<0.05).

### Experimental Example 3

### Effect of Betaine and Lycium chinensis Leaf Extract on Liver Function

Mice administered with JBB21, LWE, and JBB21+LWE were euthanized with CO₂ gas and underwent laparotomy to excise the livers therefrom. The livers were weighed with a chemical balance, and compared with those from the control and the obese groups. A part of the liver was frozen in liquid nitrogen (LN₂) to measure triglyceride and cholesterol contents while the other part was fixed in formalin for pathological analysis.

The frozen liver tissues were homogenized by a homogenizer, followed by centrifugation. The supernatant was analyzed for hepatic triglyceride and cholesterol levels using a triglyceride quantification kit (Cat. #: K622-100), and a cholesterol/cholesteryl ester quantification kit (Cat. #: K603-100), respectively, both commercially available from Biovision.

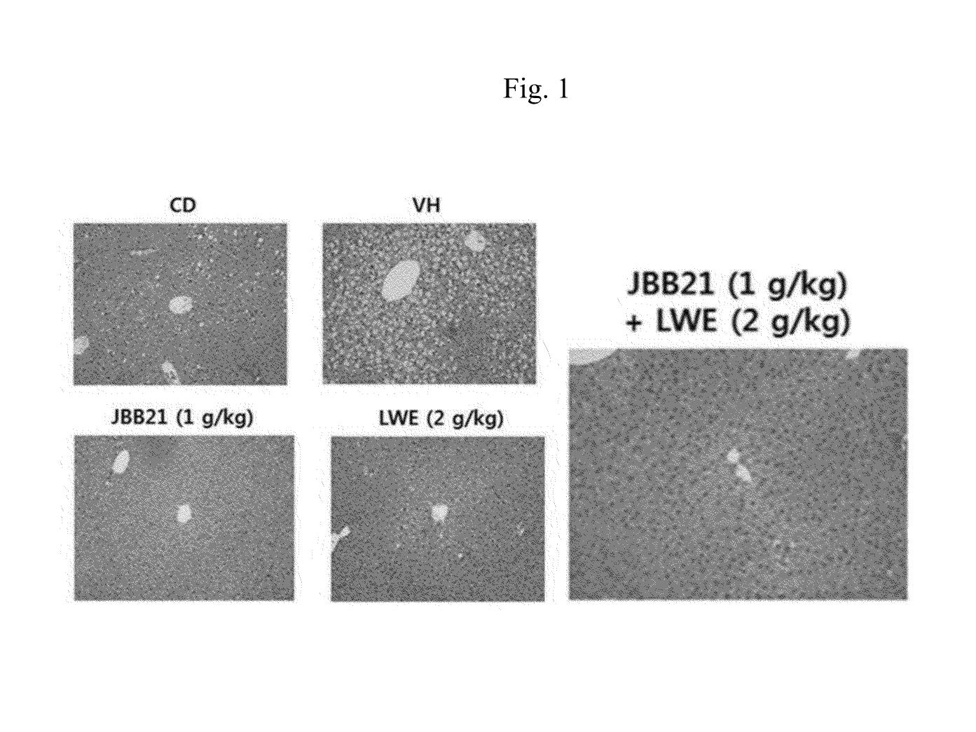

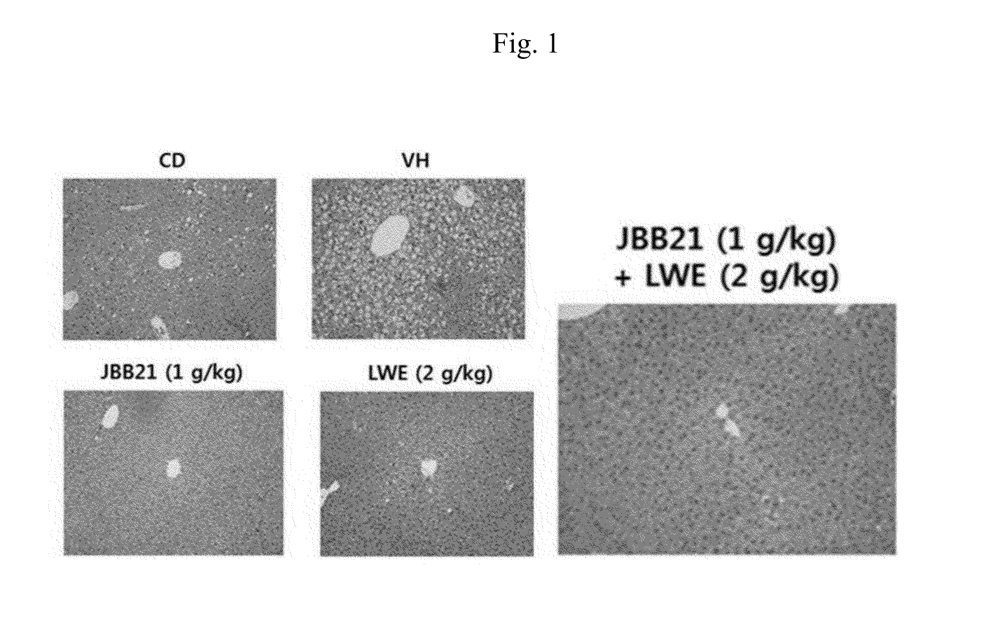

For pathological analysis, the liver tissue fixed in formalin was embedded in a paraffin block, and sectioned, and stained with H&E (Hematoxylin & Eosin) before observation under an optical microscope (FIG. 1).

An effect of the active ingredients of the present invention was evaluated in view of liver weight relative to that of the obese group (Table 7). The liver of the HFD-fed obese group was 1.4 times heavier than that of the negative control while the weight of the liver was reduced by 25.4% in the JBB21 (1 g/kg) group and by 19.4% (p<0.05) in the LWE (2 g/kg) group. Co-administration of JBB21+LWE (1+2 g/kg) reduced the weight of the liver by 26.8% (p<0.001).

TABLE 7
Effect of Betaine and/or Lycium chinensis leaf
extract on Weight and Triglyceride Level of Liver

| | | High Fat Diet (60% Kcal) | | | |
|---|---|---|---|---|---|
| | | | | | JBB21 + |
| | Control | | | | LWE |
| | Diet(10% | V.C | JBB21 | LWE | (1 + 2 |
| Group | Kcal) | (3.D.W) | (1 g/kg) | (2 g/kg) | g/kg) |
| Liver Wt | 1443.6 ± | 2026.8 ± | 1511.2 ± | 1634.2 ± | 1483.9 ± |
| (mg) | 231.7 | 353.0 | 270.9 | 413.5 | 243.7 |
| IR(%) | | | 25.4*** | 19.4* | 26.8*** |
| Hepatic | 1.0545 ± | 3.5202 ± | 3.1389 ± | 3.1185 ± | 2.3175 ± |
| triglyceride | 0.2405 | 0.6606 | 0.9591 | 0.9332 | 0.9091 |
| (nmole/ml) | | | | | |
| IR(%) | | | 10.8 | 11.4 | 34.2** |
| GOT (IU/L) | 93.8 ± | 142.1 ± | 57.1 ± | 65.7 ± | 58.0 ± |
| | 79.2 | 96.8 | 10.8 | 23.0 | 14.6 |
| IR(%) | | | 59.8** | 53.8** | 59.2** |
| GPT(IU/L) | 29.9 ± | 121.5 ± | 39.8 ± | 51.4 ± | 35.6 ± |
| | 23.5 | 120.0 | 19.2 | 36.4 | 21.7 |
| IR(%) | | | 67.2** | 57.7* | 70.7** |

*p < 0.05,
**p < 0.01,
***p < 0.001,
****p < 0.0001 vs. Vehicle Control

Administration of JBB21 (1 g/kg) or LWE (2 g/kg) alone reduced hepatic triglyceride contents by 10.8%, and 11.4%, respectively, compared to the control. In the JBB21+LWE (1+2 g/kg) group, the hepatic triglyceride content was reduced by 34.2% (p<0.01), which is three times as great as the decrements obtained by the single administration, demonstrating that the co-administration exhibited a synergistic effect on the reduction of hepatic triglyceride content.

Compared to the obese group, a GOT index, indicative of the formation of fatty liver, was also found to reduce by 59.8% (p<0.01) in JBB21 (1 g/kg), by 53.8% (p<0.01) in the LWE (2 g/kg) group, and by 59.2% (p<0.01) in the JBB21+LWE (1+2 g/kg) group.

Particularly, GPT reduced by 67.2% (p<0.01) in the JBB21 (1 g/kg) group, by 57.2% (p<0.05) in the LWE (2 g/kg) group, and by 70.7% (p<0.01) in the JBB21+LWE (1+2 g/kg) group, with statistical significance, compared to the control. Accordingly, betaine, and betaine-Lycium chinensis extract exhibited excellent preventive effects on fatty liver.

Moreover, a high triglyceride level was detected in the liver of the HFD group while the administration of JBB21, LWE, or JBB21+LWE significantly reduced hepatic triglyceride levels, exhibiting clean and healthy livers almost free of fat deposits.

### Experimental Example 4

### Statistic Analysis

Statistics was performed with the GraphPad Prism software (GraphPad Software). Weights and feed intake were compared using a two-way ANOVA, followed by the Bonferroni multiple comparison test. The other treatments were analyzed using a one-way ANOVA, followed by the Dunnett's multiple comparison test.

Preferred examples of food compositions are given as follows.

### Preparation Example 1

A health functional seasoning was prepared by mixing a commercially available, typical seasoning with 5˜7.5 wt % of a betaine powder and 10˜30 wt % of a Lycium chinensis leaf extract powder.

### Preparation Example 2

A ketchup product for body fat reduction was prepared by adding 1˜5 wt % of a betaine powder and 10˜15 wt % of a Lycium chinensis leaf extract powder to a commercially available ketchup product.

### Preparation Example 3

Flour was mixed with 1˜10 wt % of a betaine powder and 5˜15 wt % of a Lycium chinensis leaf extract powder and the mixture was used to make health functional breads, cakes, cookies, crackers, and noodles.

### Preparation Example 4

A health functional soup product was prepared by adding 1˜10 wt % of a betaine powder and 5˜10 wt % of a Lycium chinensis leaf extract powder to a commercially available soup product.

### Preparation Example 5

A health functional beef product was prepared by adding 1˜10 wt % of a betaine powder and 5˜20 wt % of a Lycium chinensis leaf extract powder to a commercially available beef product.

### Preparation Example 6

Milk was mixed with 1˜10 wt % of a betaine powder and 1˜20 wt % of a Lycium chinensis leaf extract powder, and the mixture was used to make health functional dairy foods, such as butter, ice cream, cheese, etc.

### Preparation Example 7

A carbonated beverage was prepared by adding 0.1˜10 wt % of a betaine powder and 1˜10 wt % of a Lycium chinensis leaf extract powder to a commercially available carbonated beverage.

### Preparation Example 8

A fruit juice containing 0.1˜10 wt % of a betaine powder and 1˜10 wt % of a Lycium chinensis leaf extract powder was prepared.

### Preparation Example 9

A vegetable juice containing 0.1˜10 wt % of a betaine powder and 5˜20 wt % of a Lycium chinensis leaf extract powder was prepared.

### Preparation Example 10

A health functional alcoholic beverage was prepared by adding 0.1˜10 wt % of a betaine powder and 5˜15 wt % of a Lycium chinensis leaf extract powder to a commercially available alcoholic beverage.

### INDUSTRIAL APPLICABILITY

As described hitherto, betaine and a Lycium chinensis leaf extract powder or a Lycium chinensis leaf hot water extract, when used alone or in combination, exhibit excellent anti-obesity and hepatoprotective activities, and thus the composition comprising them as active ingredients according to the present invention is effectively used as a food or pharmaceutical composition for the prevention or treatment of obesity or obesity-induced metabolic syndrome, finding various applications in food and pharmaceutical fields.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A pharmaceutical composition for preventing and treating obesity or obesity-induced metabolic syndrome, comprising a betaine compound represented by the following Chemical Formula 1 as an active ingredient:
(CH₃)₃N⁺—CH₃—COO⁻ <Chemical Formula 1>

2. The pharmaceutical composition of claim 1, wherein the betaine compound is in a form selected from the group consisting of an anhydride, a hydrate, or a pharmaceutically acceptable salt.

3. A health functional food composition for improving obesity or obesity-induced metabolic syndrome, comprising the betaine compound of claim 1 as an active ingredient.

4. A pharmaceutical composition for preventing and treating obesity or obesity-induced metabolic syndrome, comprising a mixture of a Lycium chinensis leaf extract powder and a betaine powder of claim 1 at a ratio of 1˜5:1.

5. The pharmaceutical composition of claim 4, wherein the Lycium chinensis leaf extract powder is selected from the group consisting of a powder prepared from a hot water extract of Lycium chinensis leaves, a powder prepared from an ethanol extract of Lycium chinensis leaves.

6. The pharmaceutical composition of claim 4, further comprising a Lycium chinensis leaf extract powder, tannin, hydroxyl citrate, catechin, polyphenol, bioflavonoid, a vitamin complex, taurine, amino acids, choline, l-carnitine, alone or in combination.

7. The pharmaceutical composition of claim 6, wherein the vitamin complex comprises at least one selected from vitamins A, B1, B2, B6, B12, and E, nicotinic acid amide, calcium pantothenic acid, and folic acid.

8. A health functional food composition for improving obesity, hyperlipidemia or fatty liver, comprising the composition of claim 4.

9. A pharmaceutical composition for preventing and treating obesity, hyperlipidemia or fatty liver, comprising the composition of claim 4.
